(19) United States
(12) Patent Application Publication
(10) Pub. No.: US 2026/0198977 A1
(43) Pub. Date: Jul. 16, 2026

(54) SET FOR TREATING BONE FRACTURES WITH AN H-SHAPED TOOL

(71) Applicant: I.T.S. GmbH, Lassnitzhöhe (AT)

(72) Inventors: Ronald PRAGER, Bovenau (DE); Thomas RESEDARITZ, Großhartmannsdorf (AT)

(73) Assignee: I.T.S. GmbH, Lassnitzhöhe (AT)

(21) Appl. No.: 19/446,442

(22) Filed: Jan. 12, 2026

(30) Foreign Application Priority Data
Jan. 14, 2025 (AT) A 50017/2025

(51) Int. Cl. A61B 17/74; A61B 17/90; A61B 17/56

(52) U.S. Cl. CPC A61B 17/744; A61B 17/90; A61B 2017/564

(57) ABSTRACT

A set for treating bone fractures, in particular proximal femur fractures, a tool for use with the set, and a combination of the set and tool. The set includes a bone nail; a bone screw; and an adjustment screw. The bone nail has an opening through which the bone screw is guidable, and the adjustment screw is configured to be brought into contact with the bone screw in a contact volume in order to determine a freedom of movement of the bone screw. The set also includes a tool for positive-locking alignment of the bone screw positioned on a drill wire relative to an additional drill wire.

### CROSS-REFERENCE TO RELATED APPLICATION

This application claims priority under 35 U.S.C. § 119(a) to Austria Application No. A50017/2025 filed January 14, 2025, the disclosure of which is expressly incorporated by reference herein in its entirety.

### BACKGROUND

### 1. Field of the Invention

Embodiments relate to a set for treating bone fractures, in particular proximal femur fractures, that includes a bone nail, a bone screw, and an adjustment screw. The bone nail has an opening through which the bone screw can be guided, and the adjustment screw can be brought into contact with the bone screw in a contact volume in order to determine the freedom of movement of the bone screw.

Furthermore, embodiments relate to a tool for a set for treating bone fractures, in particular proximal femur fractures. The set includes a bone nail, a bone screw, and an adjustment screw. The bone screw and the adjustment screw can be guided through an opening in the bone nail in order to interact with each other in a contact volume.

In yet another aspect, embodiments relate to a combination of a tool of the above-mentioned type and a fastening instrument for a bone screw.

Finally, embodiments relate to a surgical procedure for treating bone fractures, in particular proximal femur fractures, in which a set and/or tool of the aforementioned type is used in particular.

### 2. Discussion of Background Information

Proximal femur fractures are complicated fractures that occur particularly in older people. In addition to the complexity of the fractures, a particular problem is that bone healing takes a long time due to the average age of the patients, and therefore particularly precise but also flexible treatment of a fracture is desirable. In addition to relatively simple fixation systems, more advanced systems for treating such femoral neck fractures have become known in recent years, which, in addition to a necessary bone nail, in particular an intramedullary nail, includes a bone screw and an adjustment screw that interacts with the bone screw. In simple embodiments, the adjustment screw can engage with the bone screw such that, after fixation in a femoral head, the bone screw is fixed with regard to rotation, but also lateral displacement and medial displacement. However, systems are also known from the applicant in which, in addition to rotational stability due to interaction between the adjustment screw and the bone screw, a predetermined lateral mobility of the bone screw is provided, which is predetermined or can be predetermined according to the setting of the adjustment screw or a stop of the bone screw on the adjustment screw. In this context, systems are also known in which a desired lateral displacement is initiated on the bone screw before the healing process or immediately during the operation, thereby applying compression to the fracture fragments, in particular the femoral head and/or femoral neck and femur, such that the femoral head can be deliberately pulled laterally in order to compress bone fragments.

A set of the type mentioned above, including a bone screw and an adjustment screw, in addition to a bone nail, may be designed to provide dynamic treatment for a femoral neck fracture, whereby the adjustment screw is geometrically designed in such a way as to enable dynamic movement of the bone screw, particularly in the lateral direction. For this purpose, the bone screw usually has one or more elongated grooves, wherein the adjustment screw engages one of the grooves at a slight angle. The adjustment screw engages with the bone screw, wherein both screws, i.e., both the bone screw and the adjustment screw, can be guided through an opening in the bone nail, initially preventing rotation of the femoral head fixed with the bone screw. The groove or the (plurality of) grooves represent a contact volume into which the adjustment screw can engage. If the adjustment screw as such additionally provides lateral obstacles for the bone screw, a predetermined lateral movement of the bone screw and thus of the femoral head towards the thigh shaft can also be set in suitable geometric coordination with the bone screw.

To insert a bone screw and also an adjustment screw, appropriate drill wires are usually placed, which should run parallel (superior and inferior drill wires). The process of treating a proximal femur fracture therefore initially comprises inserting a bone nail into the medullary cavity of the thigh bone, for which a so-called targeting device is used. If necessary, the bone nail can also be hammered in slightly. The targeting device is roughly crescent-shaped and has a receptacle for a master sleeve and one or more tissue protection sleeves, which are inserted after the tissue has been cut in order to provide, for example, transverse (usually at an angle of approximately 100° to 140°) guide sleeves for drilling wires for the bone screw itself (usually superiorly positioned drill wire) and to ensure rotational stability during the insertion of the cannulated bone screw (inferiorly positioned drill wire). In the next step, the corresponding drill wires are set and then the bone screw is first driven into the femoral head, wherein the superior drill wire serves as a guide for the cannulated bone screw. The bone screw is driven forward using a fastening instrument such as a screwdriver, specifically by rotation. The bone screw, which is threaded at the medial end, is thereby driven into the femoral head. Care must be taken to ensure that the bone screw is fastened such that its groove or grooves (usually, for example, four grooves are provided at an angle of 90° relative to the circumference) are positioned such that the drill bit required for the adjustment screw, which is distal or inferior, does not collide with the bone screw during drilling and, ultimately, the adjustment screw can also be positioned within a contact volume. If the bone screw is twisted in this regard, the drill for the adjustment screw will initially collide with the bone screw during the surgical procedure or cannot be moved further. Even if the drill is still continuous, a situation may arise during the further course of the surgical procedure in which the adjustment screw cannot be perfectly inserted into a contact volume, which is essentially defined by a groove in the bone screw.

In order to enable particularly accurate alignment in this regard, it may be provided that the fastening instrument for the bone screw has a marking and that this is also the case for a sleeve surrounding the bone screw to be driven in, which also directly or indirectly determines the alignment of an inferior drill wire. If these two markings are aligned laterally, a position is given in which the bone screw or a groove of the bone screw (depending on how many there are) should be positioned so that first the drill and then the adjustment screw can be guided at a distal distance in order to achieve the desired interaction between the adjustment screw and the bone screw in the contact volume without complications arising beforehand. However, in practice, after the alignment of the fastening instrument with the corresponding marking, for example on a guide sleeve, the patient being operated on is repositioned or the tissue protection sleeve, which bears a corresponding mark, for example, experiences a slight displacement, so that the bone screw is no longer suitably aligned with its fastening volume. This can mean that a drill cannot be used to make a hole for the adjustment screw, but instead hits the bone screw or causes problems when adjusting the adjustment screw. This is undesirable during the surgical procedure because it can not only lead to longer operating times, but also to additional work for the surgical staff, as the situation differs from the norm.

### SUMMARY

Embodiments develop a set of the type mentioned at the outset, which is already very well designed in itself, such that the above complications are also prevented or at least reduced.

Another goal of the invention is to provide a suitable tool of the type mentioned above.

Finally, further embodiments provide a combination of a tool of the type mentioned above and a fastening instrument for a bone screw, which eliminates or at least reduces the problems described above.

The task of the invention relating to a set is solved when, in a set of the type mentioned at the outset, the set includes a tool for the positive-locking alignment of the bone screw positioned on a drill wire relative to an additional drill wire.

Such a set offers the advantage that the bone screw is positioned precisely in relation to the drill wire passing through the opening and thus also to the adjustment screw to be inserted later. The adjustment screw can therefore be easily inserted and engage in a contact volume defined in particular by the bone screw. For example, the contact volume can be a recess such as a groove or a plurality of grooves distributed evenly around the circumference of the bone screw, but also other geometric shapes such as tiered shapes. The positive-locking alignment and connection of the bone screw with the additional (inferior) drill wire ensures intraoperative rotational stability of the bone screw relative to the drill wire even without inserting the adjustment screw. This means that during this positive-locking connection, even when patients are repositioned during an operation, a bone screw that was initially aligned correctly by hand cannot change position, for example because the patient is being handled or because the patient’s fascia and the tensile and/or compressive forces exerted by it cause the tissue protection sleeve to shift.

The tool usually connects the bone screw and the other (inferior) drill wire indirectly. In particular, the set may include a guide, in particular a sleeve, for the additional drill wire. The guide accommodates the tool for aligning the bone screw in a positive-locking manner and vice versa. For this purpose, the tool usually engages with the guide on the outside. For the counterpart of the positive-locking connection, it may be provided that the set includes a rotator for the bone screw, wherein the tool is displaceable, in particular, linearly displaceable, mountable and/or mounted on the rotator. The rotator, which is shaped like a screwdriver, for example, is designed to screw the bone screw into the drill wire provided for the bone screw, thereby determining the correct position of the bone screw using markings, such that after removal of the inferior drill wire and the sleeve for it, a guide sleeve for the drill can first be inserted into a master sleeve and/or tissue protection sleeve and the drill for the adjustment screw can be guided past the bone screw during drilling, and then the adjustment screw is correctly positioned in relation to the bone screw when inserted. The rotator can engage at its medial end in a lateral end of the bone screw, in particular in a positive-locking manner, such as via a hexagon. In this case, the tool can be mounted on the rotator. For this purpose, the tool can in particular be pushed onto the rotator and is advantageously mounted on it in a displaceable manner. The rotator can then be operated together with the attached tool to screw the bone screw into the desired position. The tool is mounted at the end of the rotator and is inactive. Once the bone screw is in the correct position, based on human judgment and the aforementioned markings, the tool can be displaced medially from a lateral position on the rotator so that the tool rests against the rotator on one side and against a sleeve for the inferior drill wire on the other. Since the sleeve for the inferior drill wire is essentially fixed in position in an external or overlapping master sleeve, the positive-locking connection of the sleeve to the bone screw via the tool ensures an indirect positive-locking connection of the bone screw to the drill wire, wherein the bone screw is not only held in a rotationally stable manner, but is also correctly positioned. Subsequently, the rotator can be removed and the inferior drill wire and sleeve can be removed, after which the necessary hole for the adjustment screw can be drilled before the adjustment screw is inserted after the drill has been removed.

In this context, it is particularly preferable that the tool has at least one lug and the rotator has at least one corresponding indentation to accommodate the lug in the indentation. The at least one indentation can accommodate the lug in a positive-locking manner. Preferably, a plurality of lugs and a plurality of corresponding indentations are provided. If, for example, two lugs opposing each other by 180° and correspondingly two indentations opposing each other by 180° are provided on the rotator, the tool can slide optimally on the rotator and can also be easily attached and removed. Other alignments, for example at 90° or 120°, are of course also possible, but require more manufacturing effort. Incorrect placement is not possible, as a plurality of lugs ensure correct positioning of the tool on the rotator.

In order to eliminate potential sources of human error due to incorrect handling as far as possible, it is advantageous for the tool to be designed so that, when viewed from the front, it is symmetrically identical in relation to a first center axis and/or symmetrically identical in relation to a second center axis. The higher the symmetry, the less susceptible the tool is to incorrect assembly. Ideally, the tool can only be attached to a rotator in a single, simultaneously positive-locking variant, so that no error can occur in this regard.

The fastening volume is advantageously formed by at least one, preferably a plurality of, grooves in the bone screw. A plurality of fastening volumes, in particular grooves, on or in the bone screw are useful so that the insertion depth for the bone screw, in particular into a femoral head, can be designed flexibly and the bone screw does not have to be inserted too far, for example, so that the adjustment screw can engage in a single existing groove.

It is advantageous to have a first marking on a sleeve, in particular a guide sleeve, and a further marking corresponding to this, which is attached to a screw-in means for the bone screw and/or to the bone screw itself, wherein, when the markings are in alignment, the contact volume for the adjustment screw and the bone screw are aligned. This corresponds to the usual manual adjustment before the tool according to the invention for optimized alignment is activated, in particular by moving an H-shaped tool, which is held on a rotator in a manner that allows linear displacement, from a lateral (end) position to a medial position, in particular to a medial end position, in order to establish the described positive-locking connection.

It is advantageous for the adjustment screw to be guided obliquely to the bone screw through the same opening in order to engage in the contact volume, wherein the angle between the adjustment screw and the bone screw is 100° to 140°, preferably 105° to 135°, in particular 110° to 130°. The oblique (and therefore non-coaxial) positioning of the adjustment screw relative to the bone screw allows the adjustment screw to engage in a contact volume in the bone screw, wherein the corresponding positioning can also be designed in such a way that the bone screw can be moved laterally during the healing process until it strikes the adjustment screw, for example by a medially tapered groove design. If the bone screw moves laterally during the healing process and the adjustment screw is within the contact volume without initially touching the bone screw, the bone screw can move laterally until the taper of the groove causes the bone screw to rest against the adjustment screw, preventing further lateral movement of the bone screw. Regardless of this, the presence of one end of the adjustment screw in the contact volume during the healing process also prevents rotation of the bone screw, as this is blocked by the adjustment screw.

The adjustment screw usually has an external thread, and the opening may have an internal thread to accommodate the external thread of the adjustment screw. This allows the adjustment screw to be held in position in the opening of the bone nail. The bone screw, on the other hand, is fastened in a bone part such as a femoral head via an external thread, but has no external thread in the area of the opening and no internal thread in the area of the guide through the opening, so that the bone screw is mounted in the opening of the bone nail in a sliding manner or can slide in it.

In a further aspect, the invention relates to a tool of the type mentioned above, wherein the tool has a guide opening in a front view, so that the tool can be applied to a fastening instrument for the bone screw, such as a rotator. Such a tool makes it easy to ensure that, when inserting a bone screw, it can be positioned correctly and held stable until work is required at another position, in particular work to insert a drill for the subsequent insertion of an adjustment screw.

The tool can be designed in a U-shape or, preferably, an H-shape when viewed from the front. A high degree of symmetry ensures that no handling errors can occur during a stressful phase of an operation due to incorrect placement of the tool on a screwdriver.

In accordance with the above advantages of a set according to the invention and a tool designed for this purpose, the invention comprises, in a further aspect, a combination of a tool according to the invention and a fastening instrument for a bone screw, wherein the fastening instrument is configured to accommodate the tool in a positive-locking manner in the area of the guide opening. In particular, the tool can be mounted or stored on the fastening instrument in a manner that allows linear displacement.

In a further aspect, the invention relates to a surgical procedure of the type mentioned at the outset, wherein a set and/or a tool according to the invention is used.

In a further aspect, the invention relates to a surgical procedure for treating bone fractures, in particular proximal femur fractures, wherein a bone screw inserted into a bone, in particular the femoral head, is aligned with a tool by means of a positive-locking engagement of the tool on a guide for an additional drill wire while simultaneously connecting the tool to the bone screw. The additional drill wire is different from a drill wire for the bone screw. In particular, it may be a drill wire arranged inferior to the drill wire for the bone screw. Thus, the bone screw is inserted via the superiorly arranged drill wire and then aligned with the preferably parallel drill wire or a guide for the same. This ensures that the bone screw is positioned so that it can interact with a subsequently inserted adjustment screw even if the guide for the bone screw is not ideally positioned. Correct alignment is particularly necessary if the adjustment screw engages with the outside of the bone screw, especially if the adjustment screw engages with the bone screw at an angle and thus not coaxially and, if necessary, engages in a groove of the bone screw.

In particular, it may be provided that the tool is connected to the bone screw indirectly, in particular via a screwdriver for the bone screw. The screwdriver can be brought into engagement with a lateral end of the bone screw with a medial end thereof, wherein the tool is mounted on the screwdriver and displaced from a lateral position to a medial position, in each case on the screwdriver, in order to align the bone screw. The alignment of the bone screw can be effected by rotation of the bone screw. The bone screw is rotated about its longitudinal axis.

In general, the surgical procedure described above may include several or all of the following features, which are preferably performed in the order listed:

connecting and inserting the bone nail with the targeting device;

arranging a master sleeve;

inserting a guide for a superior drill wire and a guide for an inferior drill wire, after which the drill wires are inserted, preferably in parallel;

pre-drilling for a cannulated bone screw, in particular a bone screw with external grooves;

inserting the bone screw via the superior drill wire, in particular with a screwdriver attached laterally to the bone screw;

aligning the bone screw relative to the inferior drill wire;

removing the inferior drill wire;

inserting an inferior guide sleeve for a drill bit to drill a hole for an adjustment screw;

pre-drilling for the adjustment screw;

removing the drill bit and inserting the adjustment screw, wherein the adjustment screw engages in particular in a groove of the bone screw.

In embodiments, a set for treating bone fractures, in particular proximal femur fractures, can include a bone nail; a bone screw; and an adjustment screw. The bone nail can have an opening through which the bone screw is guidable, and the adjustment screw may be configured to be brought into contact with the bone screw in a contact volume in order to determine a freedom of movement of the bone screw. The set further includes a tool for positive-locking alignment of the bone screw positioned on a drill wire relative to an additional drill wire.

In other embodiments, a tool for a set for treating bone fractures, in particular proximal femur fractures, in which the set includes a bone nail, a bone screw, and an adjustment screw, and the bone screw and the adjustment screw are guidable through an opening in the bone nail in order to interact with each other in a contact volume, the tool can include a guide opening when viewed from a front surface of the tool, the guide opening being configured to apply the tool to a fastening instrument for the bone screw, such as a rotator.

According to still other embodiments, a surgical procedure for treating bone fractures, in particular proximal femur fractures, may use at least one of: a set that includes a bone nail, a bone screw, and an adjustment screw, and the bone screw and the adjustment screw are guidable through an opening in the bone nail in order to interact with each other in a contact volume, and/or a tool that includes a guide opening when viewed from a front surface of the tool, the guide opening being configured to apply the tool to a fastening instrument for the bone screw, such as a rotator.

In accordance with still yet other embodiments, a surgical procedure for treating bone fractures, in particular proximal femur fractures, may include guiding a bone screw into a bone, in particular the femoral head, in which the bone screw is aligned with a tool via a positive-locking engagement of the tool on a guide for an additional drill wire while simultaneously connecting the tool to the bone screw.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a set according to the invention with a tool in a first, lateral position of the tool;

FIG. 2 shows a rear view of the set from FIG. 1;

FIG. 3 shows a perspective view of the set from FIGS. 1 and 2;

FIG. 4 shows the set from FIGS. 1 to 3 with the tool in a medial position;

FIG. 5 shows a rear view of the set from FIG. 4;

FIG. 6 shows a front view of the set from FIGS. 4 and 5;

FIG. 7 shows a perspective view of the set from FIGS. 4 to 6;

FIG. 8 shows a frontal view of a tool for a set according to embodiments;

FIG. 9 shows a side view of the tool from FIG. 8;

FIG. 10 shows a top view of a tool according to FIG. 8;

FIG. 11 shows a perspective view of a tool according to FIGS. 8 to 10;

FIG. 12 shows a schematic illustration of a fixed bone nail with bone screw and adjustment screw.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

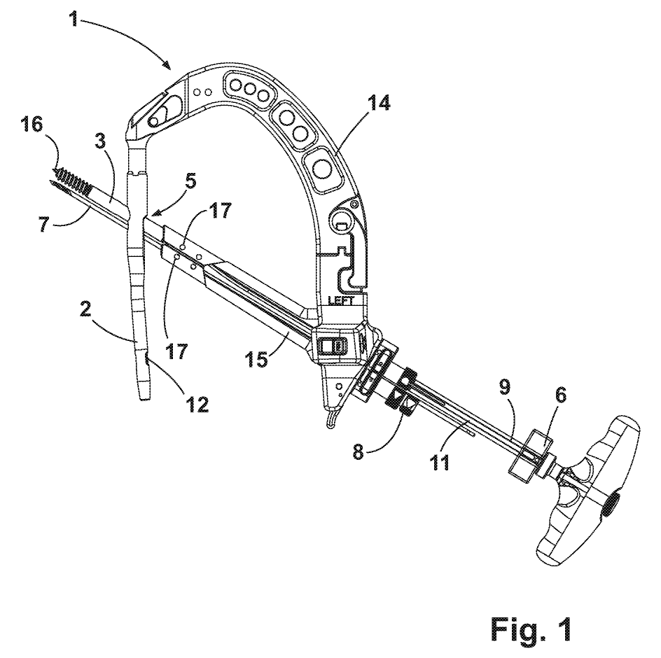

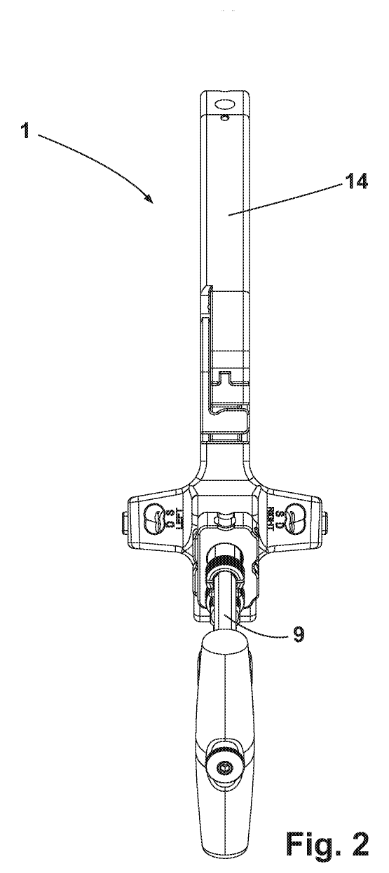

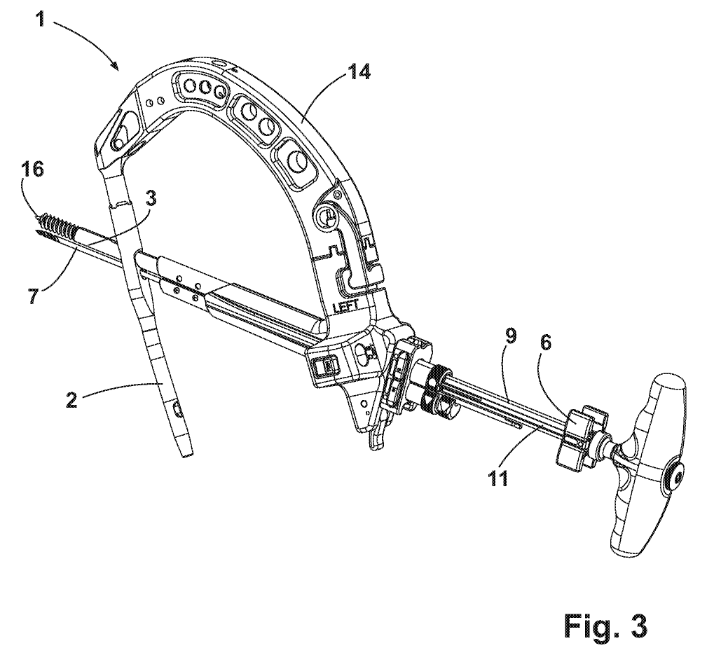

FIG. 1 to FIG. 3 show various views of a set 1 according to the invention. Set 1 comprises a targeting device 14. The targeting device 14 can be connected to a visible bone nail 2 in order, on the one hand, to be able to insert the bone nail 2, in particular into a femur in the case of a femoral neck fracture. Furthermore, the targeting device 14 also accommodates a master sleeve 15 opposing the bone nail 2, which in particular also serves to further accommodate one or more tissue protection sleeves. The master sleeve 15 is guided through an opening in the targeting device 14 so that the master sleeve 15 is directed at least substantially toward an opening 5 in the bone nail 2. This ensures that parts to be inserted later are always positioned correctly. Inside the master sleeve 15 and, if applicable, a tissue protection sleeve, there is first a guide (no longer visible in FIG. 1) for a drill wire 16 and a guide 8 for an additional drill wire 7 running parallel to it. The drill wire 16 acts as a guide for a bone screw 3, which is a cannulated bone screw 3 and is guided over the drill wire 16. Both guide 8 and the other guide can be designed as insertable sleeves. The bone screw 3 is inserted via the drill wire 16 and driven into a femoral head in particular. Regardless of a tool 6, which is particularly visible in FIG. 1 and FIG. 3 and which is located in FIG. 1 to FIG. 3 in a lateral position, in particular in a lateral end position, on a rotator 9 in the form of a screwdriver for the bone screw 3, the situation according to FIG. 1 to FIG. 3 represents a situation in a typical surgical procedure. The course of the operation is general and, detached from the specific exemplary embodiment, even without the tool 6, is in principle as follows:

connecting and inserting the bone nail 2 with the targeting device 14;

positioning the master sleeve 15 and optional tissue protection sleeves;

inserting a guide for the superior drill wire 16 and a guide 8 for the inferior drill wire 7, wherein the drill wires 7, 16 are preferably inserted parallel;

pre-drilling for the cannulated bone screw 3;

inserting the bone screw 3 via the drill wire 16 using a screwdriver attached laterally to the bone screw 3;

removing the inferior drill wire 7;

inserting an inferior guide sleeve for a drill bit to drill a hole for the adjustment screw 4;

pre-drilling for the adjustment screw 4;

removing the drill bit and inserting the adjustment screw 4.

In addition, the usual steps required to perform the next step must be carried out, for example, the removal of individual guides 8. Window 17 can be used to check the alignment of the drill wires 7, 16.

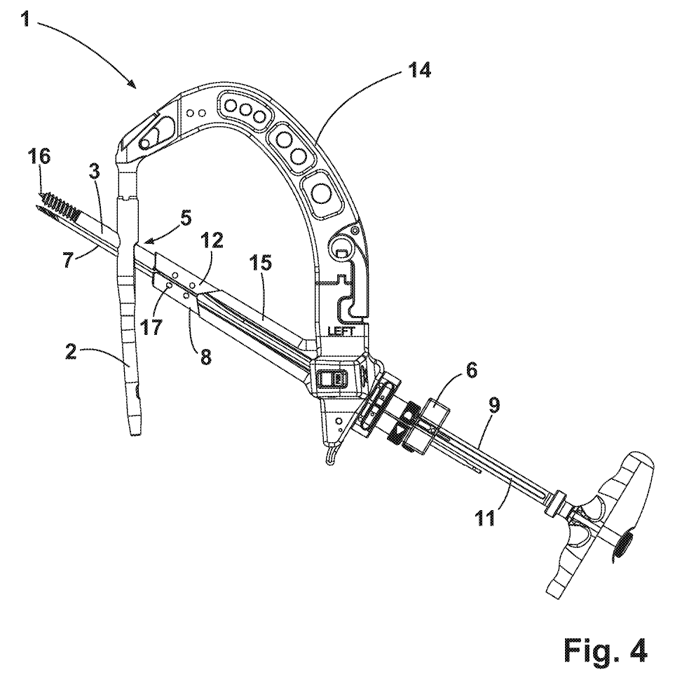

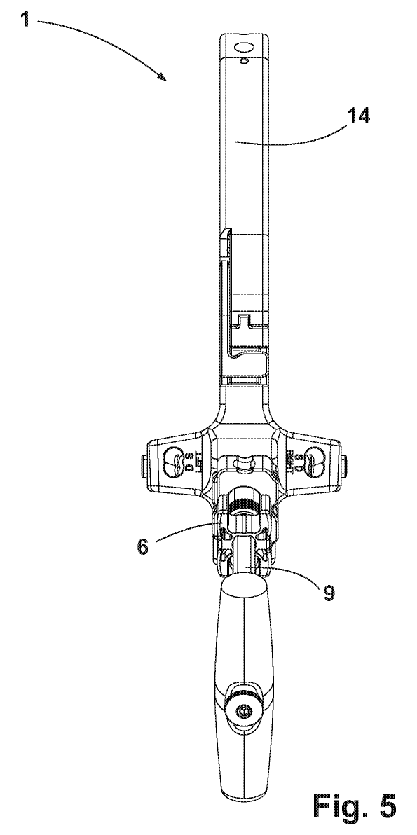

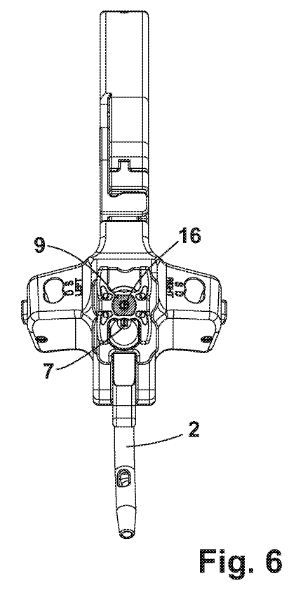

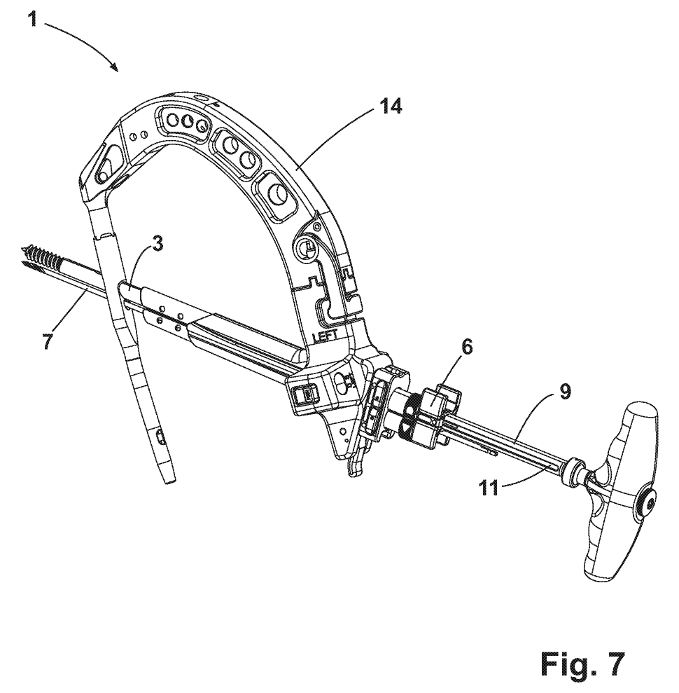

When inserting the bone screw 3, it is necessary to ensure that the bone screw 3 is correctly positioned, especially if it is designed with lateral grooves to define a contact volume for an adjustment screw 4 (for example, four external grooves that are distributed equidistantly around the circumference at 90° intervals). If this is not the case, the drill for the adjustment screw 4 cannot be guided past the bone screw 3 in a suitable manner, but instead collides with the bone screw 3. For this reason, the rotator 9 or screwdriver for the bone screw 3 has a marking, in particular a straight line, which must be aligned with a marking on a tissue sleeve seated in the master sleeve 15 or another part of the set 1. The marking can be located not only on the screwdriver, but alternatively or additionally also on the bone screw 3. Preference is given to straight lines as the markings to be aligned, so that when aligned, the markings are in a straight line. It can then be assumed with a relatively high degree of certainty that the manual adjustment process for positioning the bone screw 3 is suitable for first performing the drilling process for the adjustment screw 4 and then positioning the adjustment screw 4 appropriately. However, during surgery, for example, due to repositioning of the patient or minor displacement of a guide sleeve with a small amount of play of a few degrees, the correct positioning of the bone screw 3 relative to the drill wire 7 may no longer be maintained. To prevent this, a tool 6 is provided which, in FIGS. 1 to 3, remains in a lateral position, in particular a lateral end position, on the rotator 9 until the rotator 9 has brought the bone screw 3 into a desired position (alignment of the markings). Later during the operation, the tool 6 is moved linearly medially on the rotator 9 until the tool 6 comprises the guide 8, as shown in FIGS. 4 to 7. This provides a positive-locking connection and ensures that the bone screw 3 is correctly aligned relative to the drill wire 7. This minimizes the risk of losing the alignment of the bone screw 3 relative to the drill wire 7 and thus ultimately or subsequently also to the adjustment screw 4 due to repositioning of the patient or other manual techniques. Once the correct setting has been achieved, the tool 6 can be removed and the inferior drill wire 7 and associated sleeve can be removed. The drill for the adjustment screw 4 can now be guided in, after which the adjustment screw 4 can be inserted.

The method, as previously defined in general terms, thus additionally comprises, according to the invention, displacement of the tool 6 to correctly align the bone screw 3 after its insertion, but before drilling a hole for the adjustment screw 4.

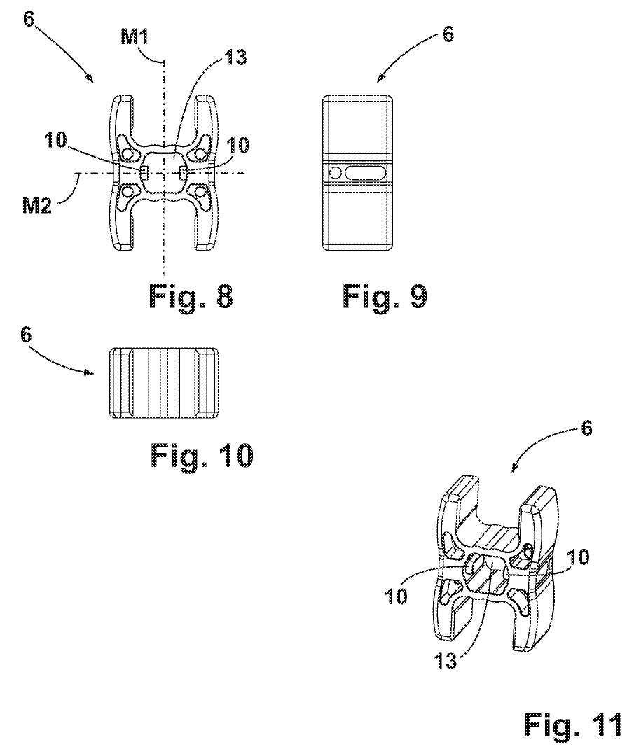

FIG. 8 to FIG. 11 show a tool 6 according to the invention separately in different views. As can be seen in particular from FIG. 8 and FIG. 11, the tool 6 is essentially H-shaped. The tool 6 has a high degree of symmetry. The tool 6 is designed to be symmetrically identical in relation to a first center axis M1 and a second center axis M2. This results in two-fold rotational symmetry around the corresponding axes (symmetrical axis of rotation of 180°), which means that the tool 6 cannot be mounted incorrectly on a rotator 9. Regardless of how the tool 6 is placed on a rotator 9, as shown in FIGS. 1 to 7, the tool 6 is correctly mounted for or during an operation.

With reference to FIG. 8 or FIG. 11, tool 6 has a central guide opening 13. The central guide opening 13 has two lugs 10 on the inside, which are arranged at the same height and can be inserted into corresponding indentations 11 of a fastener such as a rotator 9. This allows, on the one hand, a positive-locking attachment of the tool 6 to the fastener, such as a rotator 9, and, on the other hand, a linear displacement of the tool 6 on the rotator 9, so that the rotator 9 can be operated while the tool 6 remains in a passive position, as shown in FIG. 1 to FIG. 3. Starting from the central guide opening 13, two arms extend both upward and downward in FIG. 8 and FIG. 11, which essentially define the H-shaped configuration of the tool 6. The corresponding four arms (two arms above, two arms below) ensure that the tool 6 can interact with other parts of the set 1 according to the invention in the manner described, so that a rotationally stable alignment of a bone screw 3 relative to, in particular, a drill wire 7 can be achieved already during the performance of an operation, so that it is ensured during the operation that a subsequent insertion of an adjustment screw 4 is possible without complications.

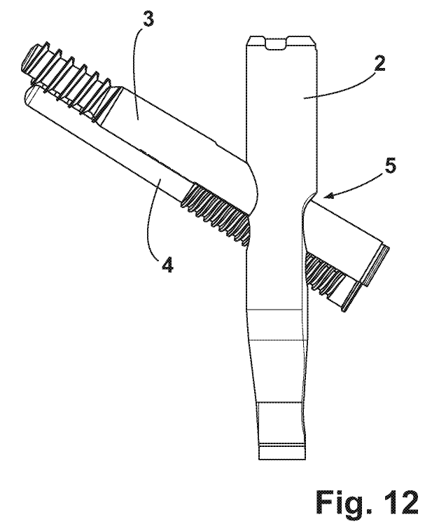

Finally, FIG. 12 shows a schematic illustration of a fixed bone nail 2 with bone screw 3 and adjustment screw 4, as it is after surgery and during the healing process. In addition, there is usually at least one fixed distal fastening of the bone nail 2 via the distal opening 12 shown in FIG. 1, for which a further screw is provided, which runs essentially horizontally with reference to FIG. 12.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A set for treating bone fractures, in particular proximal femur fractures, comprising:
a bone nail;
a bone screw;
an adjustment screw;
wherein the bone nail has an opening through which the bone screw is guidable, and
wherein the adjustment screw is configured to be brought into contact with the bone screw in a contact volume in order to determine a freedom of movement of the bone screw; and
a tool for positive-locking alignment of the bone screw positioned on a drill wire relative to an additional drill wire.

2. The set according to claim 1, wherein the tool indirectly connects the bone screw and the additional drill wire.

3. The set according to claim 1, further comprising a guide, in particular a sleeve, for the additional drill wire,
wherein the guide accommodates the tool for aligning the bone screw in a positive-locking manner.

4. The set according to claim 1, further comprising a rotator for the bone screw,
wherein the tool is displaceable, in particular linearly displaceable, mountable and/or mounted on the rotator.

5. The set according to claim 4, wherein the tool has at least one lug, and the rotator has at least one corresponding indentation to accommodate the lug in the indentation.

6. The set according to claim 5, wherein the at least one indentation accommodates the lug in a positive-locking manner.

7. The set according to claim 5, wherein the at least one lug comprises a plurality of lugs and indentations corresponding to the plurality of lugs.

8. The set according to claim 1, wherein the tool has a front surface that, when viewed from the front, is designed to be approximately H-shaped.

9. The set according to claim 1, wherein the tool has a front surface that, when viewed from the front, is configured to be symmetrically identical in relation to a first center axis and/or symmetrically identical in relation to a second center axis.

10. The set according to claim 1, wherein the contact volume is formed by at least one groove, and preferably a plurality of grooves, in the bone screw.

11. The set according to claim 1, wherein a first marking is located on a sleeve, in particular a guide sleeve, which corresponds to a further marking located on a screw-in element for the bone screw and/or the bone screw, and
wherein, when the markings are in alignment, the contact volume for the adjustment screw and the bone screw is aligned.

12. The set according to claim 1, wherein the adjustment screw is guidable obliquely to the bone screw through the opening in order to engage in the contact volume, and
wherein an angle between the adjustment screw and the bone nail is 100° to 140°, preferably 105° to 135°, and more preferably 110° to 130°.

13. The set according to claim 1, wherein the adjustment screw has an external thread and the opening has an internal thread to accommodate the external thread of the adjustment screw.

14. A tool for a set for treating bone fractures, in particular the set according to claim 1, in particular proximal femur fractures, wherein the set includes a bone nail, a bone screw, and an adjustment screw, and the bone screw and the adjustment screw are guidable through an opening in the bone nail in order to interact with each other in a contact volume, the tool comprising:
a guide opening when viewed from a front surface of the tool, the guide opening being configured to apply the tool to a fastening instrument for the bone screw, such as a rotator.

15. The tool according to claim 14, further comprising at least one lug, preferably a plurality of lugs, arranged on an inside of the guide opening.

16. The tool according to claim 14, wherein the tool viewed from the front surface is configured with a U-shape or preferably an H-shape.

17. A combination of the tool according to claim 14 and a fastening instrument for a bone screw, wherein the fastening instrument is configured to accommodate the tool in a positive-locking manner in the region of the guide opening.

18. The combination according to claim 17, wherein the tool is mounted or mountable on the fastening instrument in a manner that allows for linear displacement.

19. A surgical procedure for treating bone fractures, in particular proximal femur fractures, that uses at least one of:
a set that includes a bone nail, a bone screw, and an adjustment screw, and the bone screw and the adjustment screw are guidable through an opening in the bone nail in order to interact with each other in a contact volume, and/or
a tool that includes a guide opening when viewed from a front surface of the tool, the guide opening being configured to apply the tool to a fastening instrument for the bone screw, such as a rotator.

20. A surgical procedure for treating bone fractures, in particular proximal femur fractures, comprising:
guiding a bone screw into a bone, in particular the femoral head, in which the bone screw is aligned with a tool via a positive-locking engagement of the tool on a guide for an additional drill wire while simultaneously connecting the tool to the bone screw.

21. The surgical procedure according to claim 20, wherein the connection of the tool to the bone screw is an indirect connection, in particular via a screwdriver for the bone screw.

22. The surgical procedure according to claim 21, further comprising engaging the a lateral end of the bone screw with a medial end of the screwdriver,
wherein the tool is mounted on the screwdriver and is displaced from a lateral position to a medial position to align the bone screw.

23. The surgical procedure according to claim 21, wherein the bone screw is aligned by rotating the bone screw.
